(19) United States
(12) Patent Application Publication
(10) Pub. No.: US 2016/0045236 A1
(43) Pub. Date: Feb. 18, 2016

(54) Long Bone Screw for Minimally Invasive Surgery

(71) Applicant: Chih-Hsuan Wei, Chiayi City (TW)

(72) Inventor: Chih-Hsuan Wei, Chiayi City (TW)

(21) Appl. No.: 14/459,535

(22) Filed: Aug. 14, 2014

(51) Int. Cl. A61B 17/86

(52) U.S. Cl. CPC A61B 17/8605

(57) ABSTRACT

A long bone screw for a minimally invasive surgery contains a body including a holder, and the holder has two opposite first fences and two opposite second fences extending upwardly from the two opposite first fences; each second fence has a length extending out of a muscle tissue, the length of each second fence is within 6 to 12 cm; and the two opposite second fences one piece extend from the two opposite first fences, such that a fixing position of skeleton is marked clearly, and a size of a surgery wound is reduced.

### BACKGROUND OF THE INVENTION

1. Field of the Invention

The present invention relates to a long bone screw for a minimally invasive surgery which extends out of a muscle tissue to mark a fixing position of skeleton clearly and to reduce a size of a surgery wound.

2. Description of the Prior Art

A conventionally bone screw is fixed on skeleton in a spinal surgery, but it cannot extend of a muscle tissue to mark where the bone screw is located, so a size of a surgery opening is increased on the muscle tissue to obtain enough operation space.

With reference to FIG. 1, a conventional bone screw contains a body 10 and a shank 20, wherein the body 10 includes a holder 101 and a connecting seat 102. The holder 101 has two opposite first fences 103, and a length of each first fence 103 is within 1 to 1.5 cm, each first fence 103 has first threads 104 arranged on an inner wall thereof. The shank 20 includes a threaded section formed on an outer wall thereof and coupled with the connecting seat 102 of the body 10, a through hole 201 defined therein, and a plurality of feeding orifices 202 communicating with the through hole 201.

As illustrated in FIG. 2, in operation, the length of each first fence 103 is only 1 to 1.5 cm, so the conventional bone screw is planted into the muscle tissue completely. However, the conventional bone screw still has disadvantages as follows:

1. A size of a surgery opening is increased on the muscle tissue to obtain enough operation space.

2. The size of the surgery opening is increased on the muscle tissue, so the surgery opening heals slowly.

3. Due to the size of the surgery opening is increased on the muscle tissue, inflection risk raises.

4. The muscle tissue is damaged in the spinal surgery easily.

The present invention has arisen to mitigate and/or obviate the afore-described disadvantages.

### SUMMARY OF THE INVENTION

The primary objective of the present invention is to provide a long bone screw for a minimally invasive surgery which extends out of a muscle tissue to mark a fixing position of skeleton clearly and to reduce a size of a surgery wound.

Further objective of the present invention is to provide a long bone screw for a minimally invasive surgery which accelerates wound healing and decreases inflection risk.

Another objective of the present invention is to provide a long bone screw for a minimally invasive surgery which does not hurts a muscle tissue.

To obtain the above objectives, a long bone screw for a minimally invasive surgery provided by a first embodiment of the present invention contains: a body including a holder, and the holder has two opposite first fences and two opposite second fences extending upwardly from the two opposite first fences; each second fence has a length extending out of a muscle tissue, the length of each second fence is within 6 to 12 cm; and the two opposite second fences one piece extend from the two opposite first fences.

In a second embodiment, a long bone screw for a minimally invasive surgery contains: a body including a holder, and the holder has two opposite first fences and two opposite second fences extending upwardly from the two opposite first fences; each second fence has a length extending out of a muscle tissue, each first fence has a recess defined on an outer wall thereof, each second fence has a rib formed on an inner wall thereof and corresponding to the recess of each first fence.

### BRIEF DESCRIPTION OF THE DRAWINGS

FIG. 1 is a perspective view of a conventional bone screw for a minimally invasive surgery.

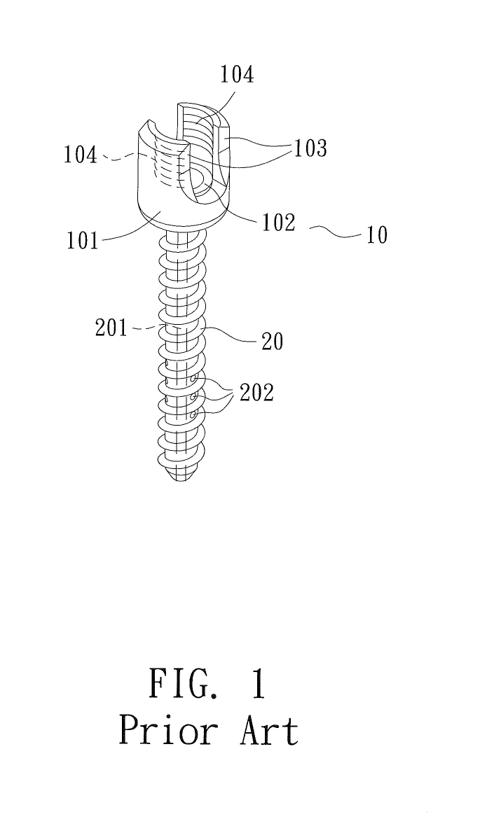

FIG. 2 is a cross sectional view showing the operation of the conventional bone screw for the minimally invasive surgery.

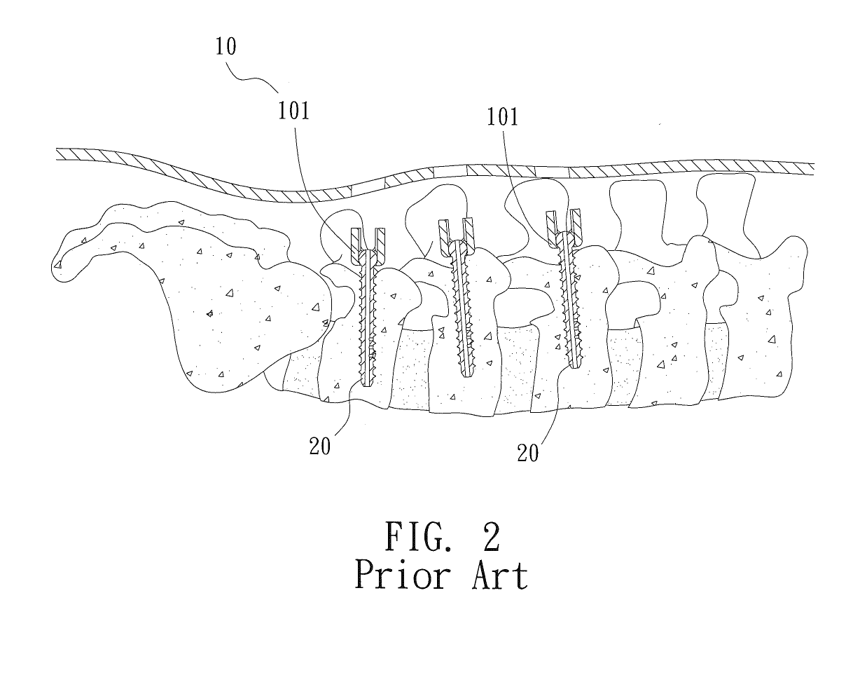

FIG. 3 is a perspective view showing the assembly of a long bone screw for a minimally invasive surgery according to a first embodiment of the present invention.

FIG. 4 is a side plan view showing the assembly of the long bone screw for the minimally invasive surgery according to the first embodiment of the present invention.

FIG. 5 is a cross sectional view showing the operation of the long bone screw for the minimally invasive surgery according to the first embodiment of the present invention.

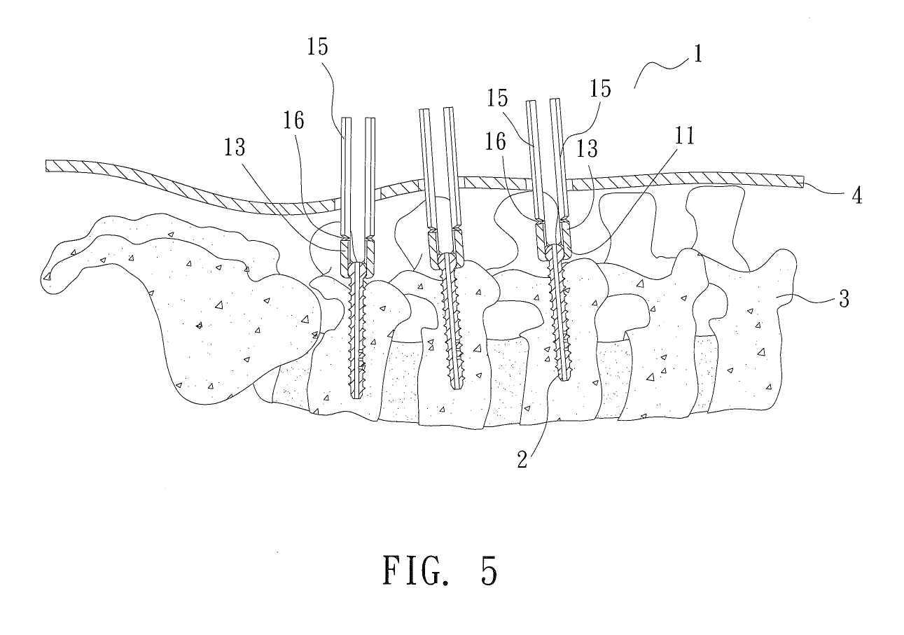

FIG. 6 is a perspective view showing the exploded components of a long bone screw for a minimally invasive surgery according to a second embodiment of the present invention.

FIG. 7 is a cross sectional view showing the exploded components of the long bone screw for the minimally invasive surgery according to the second embodiment of the present invention.

### DETAILED DESCRIPTION OF THE PREFERRED EMBODIMENTS

The present invention will be clearer from the following description when viewed together with the accompanying drawings, which show, for purpose of illustration only, the preferred embodiments in accordance with the present invention.

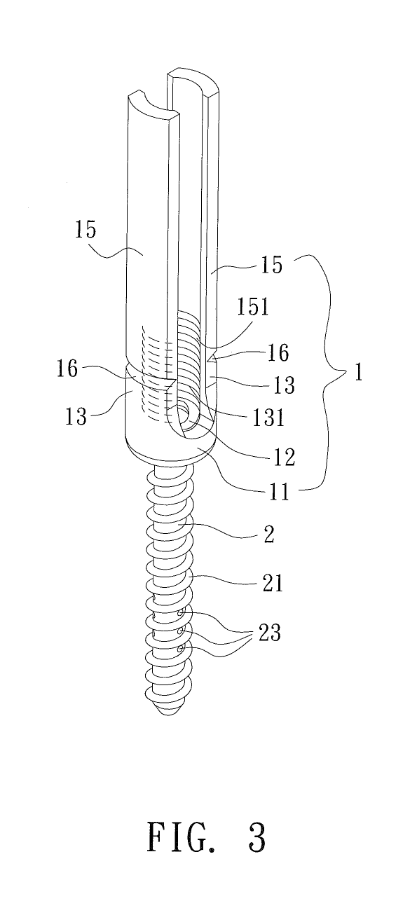

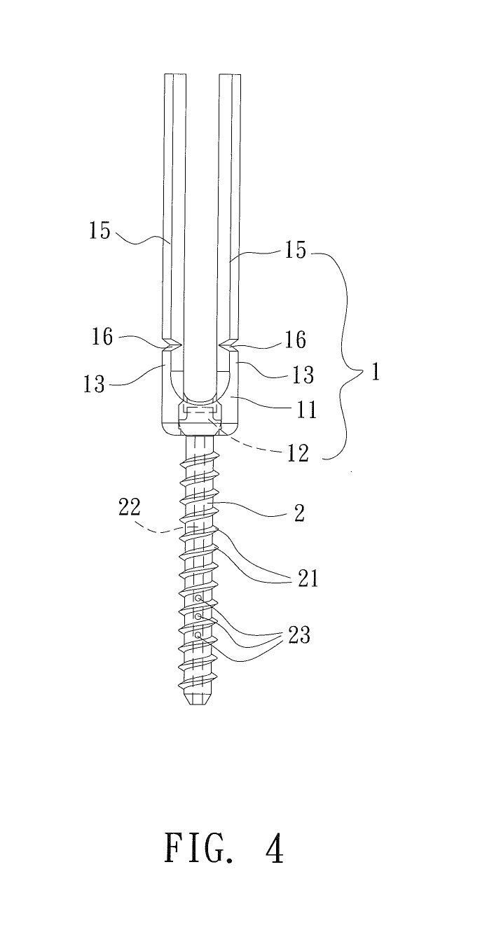

With reference to FIGS. 3 and 4, a long bone screw for a minimally invasive surgery according to a first embodiment of the present invention comprises:

a body 1 including a holder 11 and a connecting seat 12, the holder 11 having two opposite first fences 13 with a same length, and each first fence 13 having first threads 131 formed therearound;

a shank 2 including a threaded section 21 arranged around an outer surface thereof and connecting with the connecting seat 12 of the body 1, the shank 2 also including a through hole 22 defined therein and a plurality of feeding orifices 23 communicating with the through hole 22; characterized in that:

the two opposite first fences 13 of the holder 11 have two opposite second fences 15 one piece extending upwardly therefrom; each second fence 15 has a length extending out of a muscle tissue 4, and the length of each second fence 15 is within 6 to 12 cm, each second fence 15 has second threads 151 formed on an inner surface thereof;

a V-shaped slot 16 is defined between each first fence 13 and each second fence 15;

an extending direction of the first threads 131 of each first fence 13 is identical to that of the second threads 151 of each second fence 15.

Referring to FIG. 3, in a minimally invasive surgery, the shank 2 of the body 1 is screwed on a spine 3, and each second fence 15 of each first fence 13 is inserted into the spine 3 from the muscle tissue 4, such that each first fence 13 is fixed in the spine 3, and each second fence 15 exposes outside the spine 3 and extends out of the muscle tissue 4, hence medical personnel knows where the long bone screw is located by ways of each second fence 15, thus marking a fixing position of skeleton clearly. In other words, as planting the body 1, the shank 2 is screwed on the spine 3 exactly by using the body 1, thus decreasing a size of a surgery wound and inflection risk. Preferably, the V-shaped slot 16 between each first fence 13 and each second fence 15 is cut to remove each second fence 15, after finishing a minimally invasive surgery, thus leaving each first fence 13 of the body 1 in patient's body.

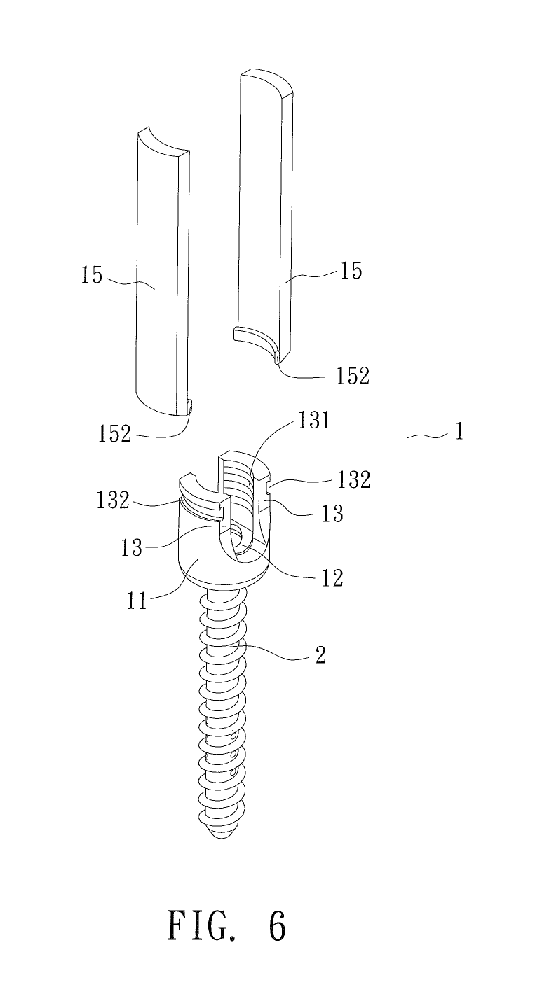

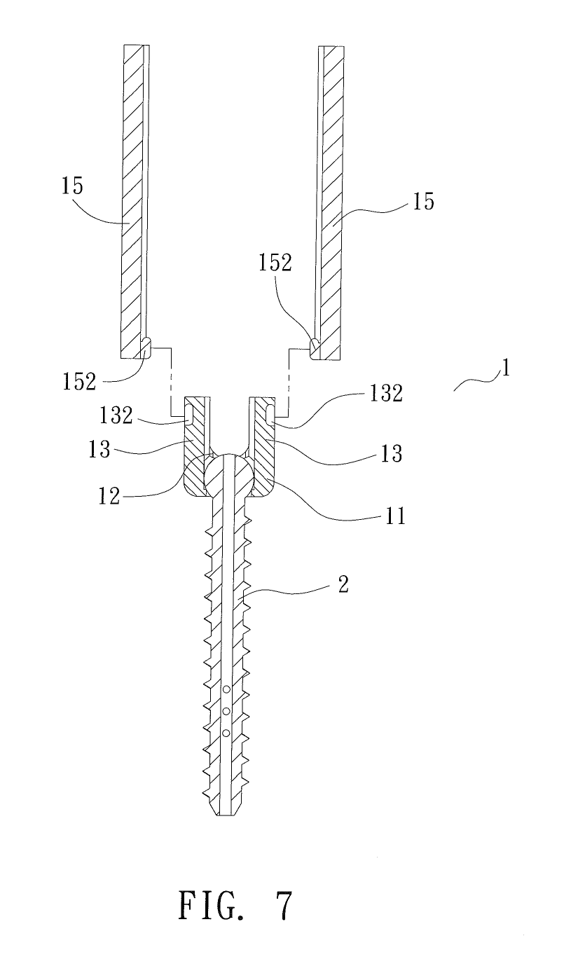

As shown in FIGS. 6 and 7, a long bone screw for a minimally invasive surgery according to a second embodiment of the present invention comprises: two opposite first fences 13 and two opposite second fences 15, and each first fence 13 has a recess 132 defined on an outer wall thereof, each second fence 15 has a rib 152 formed on an inner wall thereof and corresponding to the recess 132 of each first fence 13; and the rib 152 retains in the recess 132.

In operation, the two opposite first fences 13 and the two opposite second fences 15 are removed and fixed easily without using any tool, for example, after the long bone screw is fixed in the minimally invasive surgery, the two opposite second fences 15 are manually moved rightward or leftward, two ribs 152 of the two opposite second fences 15 are removed from two recesses 132 of the two opposite first fences 13, and the two opposite first fences 13 are removed from the two opposite second fences 15, thereby operating the long bone screw easily.

Accordingly, the long bone screw of the present invention contains the two opposite first fences 13 of the holder 11 of the body 1 and the two opposite second fences 15 one piece extending upwardly from the two opposite first fences 13, wherein the two opposite second fences 15 extend out of the muscle tissue 4 to mark the fixing position of skeleton clearly and to reduce the size of the surgery wound.

While various embodiments in accordance with the present invention have been shown and described, it is clear to those skilled in the art that further embodiments may be made without departing from the scope of the present invention.

What is claimed is:

1. A long bone screw for a minimally invasive surgery comprising:
a body including a holder, the holder having two opposite first fences and two opposite second fences extending upwardly from the two opposite first fences; each second fence having a length extending out of a muscle tissue, the length of each second fence being within 6 to 12 cm; and the two opposite second fences one piece extending from the two opposite first fences.

2. The long bone screw for the minimally invasive surgery as claimed in claim 1, wherein each second fence has second threads formed on an inner surface thereof.

3. A long bone screw for a minimally invasive surgery comprising:
a body including a holder, the holder having two opposite first fences and two opposite second fences extending upwardly from the two opposite first fences; each second fence having a length extending out of a muscle tissue, each first fence having a recess defined on an outer wall thereof, each second fence having a rib formed on an inner wall thereof and corresponding to the recess of each first fence.

4. The long bone screw for the minimally invasive surgery as claimed in claim 3, wherein the length of each second fence is within 6 to 12 cm.

5. The long bone screw for the minimally invasive surgery as claimed in claim 3, wherein each second fence has second threads formed on an inner surface thereof.
